(19) United States
(12) Patent Application Publication
(10) Pub. No.: US 2002/0155514 A1
(43) Pub. Date: Oct. 24, 2002

(54) Zinc alpha-2-glycoprotein as indicator of cancer

(76) Inventors: Laura P. Hale, Hillsborough, NC (US); David T. Price, Greenwood, LA (US)

Correspondence Address: NIXON & VANDERHYE P.C., 1100 North Glebe Road, 8th Floor, Arlington, VA, 22201, US

(21) Appl. No.: 09/998,923

(22) Filed: Dec. 3, 2001

Related U.S. Application Data
(60) Provisional application No. 60/250,159, filed on Dec. 1, 2000.

(51) Int. Cl. G01N 33/574

(52) U.S. Cl. 435/7.23

(57) ABSTRACT

The present invention relates, in general, to methods of diagnosing and monitoring cancer and inflammatory diseases/disorders and, in particular, to methods of diagnosing and monitoring cancer and inflammatory diseases/disorders that comprise assaying for elevated levels of zinc alpha-2-glycoprotein (ZAG) in serum and other body fluids. The invention also relates to methods of inhibiting thymic atrophy, including tumor-associated atrophy.

This application claims priority from Provisional Application No. 60/250,159, filed Dec. 1, 2000, the entire content of which is incorporated herein by reference.

### TECHNICAL FIELD

The present invention relates, in general, to methods of diagnosing and monitoring cancer and inflammatory diseases/disorders and, in particular, to methods of diagnosing and monitoring cancer and inflammatory diseases/disorders that comprise assaying for elevated levels of zinc alpha-2-glycoprotein (ZAG) in serum and other body fluids. The invention also relates to methods of inhibiting thymic atrophy, including tumor-associated atrophy.

### BACKGROUND

Zinc alpha-2-glycoprotein (ZAG) is a secreted 41 kDa protein first identified in human plasma in 1961 (Burgi et al, J. Biol. Chem. 236:1066-1074 (1961)). It is named for its tendency to precipitate with zinc salts and for its electrophoretic mobility that is similar to plasma α2 globulins. Immunohistochemical studies have previously demonstrated immunoreactive ZAG protein within the cytoplasm of normal secretory epithelial cells, including those in breast, prostate, and liver, as well as in salivary, bronchial, gastrointestinal, and sweat glands (Tada et al, J. Histochem. Cytochem. 39:1221-1226 (1991)). ZAG mRNA is expressed in a similar distribution, with placenta, ovary, and thyroid reportedly negative for ZAG mRNA (Freije et al, FEBS Lett. 290:247-249 (1991)). Consistent with its production by secretory epithelial cells, ZAG protein has been identified in most body fluids. The concentration of ZAG in normal human plasma or serum has been variously reported as between 25-140 μg/ml in different populations using various analytical techniques and may increase with age (Poortmans et al, J. Lab. Clin. Med. 71:807-811 (1968), Jirka et al, Clin. Chim. Acta. 85:107-110 (1978)).

The function of ZAG was unclear until recently, when Hirai et al (Hirai et al, Cancer Res. 58:2359-2365 (1998)) found that a lipid mobilizing factor isolated from the urine of human cancer patients with cachexia was identical to ZAG. Murine and human ZAG have an overall amino acid sequence identity of only 59% (Ueyama et al, J. Biochem. 116:677-681 (1994)), but share up to 100% identity in specific regions hypothesized to be important in lipid metabolism (Sanchez et al, Science 283:1914-1919 (1999)). Thus, both human and murine ZAG stimulate lipolysis in both human and murine adipocytes resulting in glycerol release and increased lipid utilization (Hirai et al, Cancer Res. 58:2359-2365 (1998)). Todorov et al (Todorov et al, Cancer Res. 58:2353-2358 (1998)) quantitated ZAG production in vitro and cachexia induction in vivo using a panel of murine tumors including the MAC16 colon adenocarcinoma, MS reticulum cell sarcoma, and B16 melanoma. The MAC16 tumor produced large quantities of ZAG and induced profound cachexia. The MS tumor did not produce ZAG and failed to induce cachexia in vivo. The B16 tumor produced approximately 20% of the ZAG produced by MAC16 tumors and caused significant loss of carcass lipid, although profound cachexia had not occurred by 8 days after tumor implantation. Tumor-produced ZAG may thus contribute to the development of cancer cachexia. Whether ZAG has additional biologic activities in addition to cachexia induction is currently unknown.

ZAG accumulates in breast cyst fluids to 30-50-fold plasma concentration (Bundred et al, Histopathol. 11:603-610 (1987), Sanchez et al, Proc. Natl. Acad. Sci. USA 94:4626-4630 (1997)) and is over-expressed in 40-50% of breast carcinomas (Bundred et al, Histopathol. 11:603-610 (1987), Sanchez et al, Cancer Res. 32:95-100 (1992), Diez-Itza et al, Eur. J. Cancer 29A:1256-1260 (1993)). Serial analysis of gene expression (SAGE) and microarray analysis have confirmed the relative over-expression of ZAG in breast cancer relative to normal mammary epithelium (Nacht et al, Cancer Res. 59:5464-5470 (1999)). In breast carcinomas, ZAG expression was found to correlate with tumor differentiation and did not independently affect prognosis (Diez-Itza et al, Eur. J. Cancer 29A:1256-1260 (1993)). ZAG has been reported to be present in normal prostate tissue (Tada et al, J. Histochem. Cytochem. 39:1221-1226 (1991)) and also to constitute 30% of the protein present in seminal fluid (Poortmans et al, J. Lab. Clin. Med. 71:807-811 (1968)).

The present invention relates to a method of screening for and/or monitoring tumor burden by measuring the level of ZAG in a body fluid. The method has application in prostate cancer as well as other cancer types. The invention further relates to methods of diagnosing inflammatory diseases or disorders associated with elevated blood levels of ZAG. Additionally, the invention relates to methods of inhibiting ZAG-induced thymic atrophy.

### SUMMARY OF THE INVENTION

The present invention relates, in general, to methods of diagnosing and monitoring cancer and inflammatory diseases/disorders and, in particular, to methods of diagnosing and monitoring cancer and inflammatory diseases/disorders that comprise assaying for elevated levels of zinc alpha-2-glycoprotein (ZAG) in serum and other body fluids. The invention also relates to methods of inhibiting thymic atrophy, including tumor-associated atrophy.

Objects and advantages of the present invention will be clear from the description that follows.

### BRIEF DESCRIPTION OF THE DRAWINGS

FIGS. 1A-1E: Immunoreactivity of normal prostate and prostate carcinomas with anti-ZAG antibody. FIG. 1A. Normal prostate acini are reactive with anti-ZAG mAb, with increased immunoreactivity in glands with strong secretory activity as indicated by dilated lumina and apocrine snouts (top of panel); FIG. 1B. Prostatic concretions are highly reactive with ZAG mAb. FIGS. 1C and 1D. The immunoreactivity pattern of prostate carcinomas ranges from global cytoplasmic (FIG. 1C) to reactivity limited to an apocrine snout pattern (FIG. 1D). The strong stromal staining seen in in highly ZAG-reactive prostate cancers (FIG. 1C) may represent “spill-over” from malignant glands. FIG. 1E. Variations in ZAG immunoreactivity within a given tumor often appear clonal and correlate with degree of tumor differentiation. Note that lower immunoreactivity is seen in the higher grade tumor (top) compared to the lower grade tumor (bottom).

FIG. 2: Serum ZAG levels are increased in patients with prostate cancer. Serum ZAG was measured by antigen capture enzyme immunoassay. Prostate cancer patients had higher serum ZAG concentrations significantly more often than the controls to which they were matched (p=0.02; see text).

FIG. 3: Thymic weight is decreased in mice bearing B16 and K1735 tumors in either subcutaneous (sq) or intracranial (ic) locations. d=day after tumor implantation. The number of animals studied is indicated on the bar.

FIG. 4: RT-PCR detection of ZAG mRNA in B16 and K1735 melanoma cells. Primers cross-intron-exon boundaries and do not amplify genomic DNA. Lanes 1=K1735; Lane 2=PCR blank; Lane 3=B16; Lane 4=100 bp markers. ZAG product is 653 bp.

FIG. 5: Western blot of recombinant human ZAG. rhZAG was purified from supernatant of ZAG-transfected 293 human kidney epithelial cells using a Ni-NTA column (Qiagen) specific for the His epitope tag. Bands were detected using the India His-Probe (Ni-HRP) reagent obtained from Pierce. Lane 1=MW markers; 2=culture supernatant; 3=flowthrough; 4=final purified ZAG; 5=control preparation purified similarly from vector tansfected cells. Similar results are seen using anti-ZAG mAbs.

FIG. 6: rhZAG is secreted by stably transfected B16 and 4TI clones. (-V=vector transfected; -Z=rh ZAG transfected) rhZAG was measured in cell culture supernatant using an antigen capture ELISA that detects only hZAG. Thus, although B16-V makes mZAG (documented by RT-PCR, see FIG. 4), the secretion of human ZAG by vector-transfected cells is zero. As noted previously, 4TI-V cells make neither mZAG nor hZAG, 10XA1, 3A2, and 10XB12 are B16-Z clones.

### DETAILED DESCRIPTION OF THE INVENTION

In one embodiment, the present invention provides a method of diagnosing cancer in a mammal. The method comprises assaying for the level of ZAG present in a biological test sample and comparing that level to a control sample, an elevated level of ZAG in the test sample being indicative of the presence of a tumor.

In the context of the present invention, diagnosing cancer includes, diagnosing the presence of the disease, monitoring the progression of the disease, monitoring the effect of any administered therapy, monitoring the recurrence of the disease after remission or surgery, and measuring any residual cancer after surgical treatment. By mammals is meant human as well as non-human mammals.

The method of the invention can be used in the diagnosis of a variety of tumor types that either produce ZAG or that occur in organs in which ZAG is normally produced. Examples include prostate tumors, breast tumors, colon tumors, squamous cell carcinomas and pancreatic tumors. The samples used can be solid (e.g., stool) or liquid. Advantageously, the sample used is a serum or plasma sample, however, other bodily fluids, such as urine, cerebrospinal fluid, seminal fluid, sweat and nipple aspirates, can also be used. In the case of serum, untreated serum can be used as can treated serum, e.g., fractionated serum in which certain components (for example, albumin) have been removed, or serum in which certain materials have been added.

The level of ZAG present in a sample (in free or complexed form) can be measured by any of a variety of suitable assays known to those skilled in the art. Such assays include immunoassays, chromatography, electrophoresis, solid phase affinity or densitometry of Western blots. Immunoassays can be performed using antibodies, polyclonal or monoclonal, against ZAG. Appropriate antibodies can be produced using standard protocols (ZAG or ZAG fragments can be used in the production of such antibodies, either isolated from natural sources or produced recombinantly). Preferred immunoassays include antigen capture (see Example below) and competitive immunoassays (for example, utilizing ZAG or a ZAG fragment bearing a detectable label). Based on the amount of ZAG that is present, it can be determined if the mammal has cancer, for example, prostate cancer, since cancer serum gives higher levels of ZAG than non-cancer serum. (Age and source matched samples can be used as controls.)

In a preferred embodiment, the present invention relates to a method of diagnosing prostate cancer. ZAG is made by normal prostate glands and malignant glands. Normal glands are connected to the ejaculatory system and the ZAG produced passes from the gland via that system and thus is not accessible to the serum. Malignant glands, however, do not connect to the ejaculatory system and growth of the tumor may disrupt the connections of normal glands to the ejaculatory system. In this case, ZAG is still produced but it cannot pass out in semen. Rather, it leaks out into the surrounding tissue, where it is picked up in lymph and from there empties into the blood, increasing the serum ZAG level. In accordance with this embodiment, prostate cancer serum can be distinguished from benign prostatic hyperplasia serum or normal serum. High levels of ZAG are present in prostate cancer serum, whereas lower levels are present in benign prostatic hyperplasia serum (or normal serum).

ZAG production is known to be induced by testosterone. A patient having elevated ZAG serum levels can be further evaluated to determine whether the elevated levels are due to the presence of cancer or simply to an enlarged normal prostate. This further evaluation can be effected by administering testosterone and thereby stimulating ZAG production. Both normal and malignant glands respond, however, only in the case of malignancy does the excess ZAG produced contribute to an elevation of the serum ZAG level. Use of such a “ZAG stimulation test” can be used to increase the specificity of the fundamental diagnostic method. The present “ZAG stimulation test” is similar to the differential response of prostate specific antigen (PSA) to testosterone surge in prostate cancer vs. benign prostatic hyperplasia reported by Agarwal et al (BJU International 85:690-695 (2000)).

Measurement of serum ZAG can be used alone as a diagnostic test or it can be used in addition to PSA level screening, or other diagnostic approach, to evaluate patients for the presence of prostate cancer.

In another embodiment, the present invention relates to a method of diagnosing or monitoring an inflammatory disease or disorder that is associated with injury to the ZAG-producing epithelium of the involved tissue or organ. The method comprises assaying for the level of ZAG present in a biological test sample and comparing that level to a control sample, an elevated level of ZAG in the test sample being indicative of the presence of an inflammatory disease or disorder, Diseases/disorders that can be detected in accordance with this embodiment include inflammation of the breast, prostate, liver or salivary, bronchial, gastrointestinal or sweat glands. Inflammatory bowel disease is a specific example of a disease detectable in accordance with this embodiment. In a preferred aspect of this embodiment, the biological sample used is a serum sample, however, other biological samples can also be used, including saliva samples. The level of ZAG present can be determined using techniques described above.

In yet another embodiment, the present invention relates to a method of inhibiting thymic atrophy in a mammal by inhibiting the deleterious effect of ZAG on thymic tissue. This method can be used in the treatment of adults experiencing thymic atrophy, for example, as a result of age, tumor, cancer chemotherapy or infection (including HIV infection). The method can be effected by administering an agent that reduces the bioavailability of ZAG and/or blocks the binding of ZAG to its receptor. Examples of such agents include anti-ZAG antibodies and anti-androgens. Optimum dosing regimens can be readily established by one skilled in the art and can vary, for example, with the agent, the patient and the effect sought. Any of a variety of routes of administration can be used, including, but not limited to, injection (e.g., IV) and oral administration.

Certain aspects of the invention can be described in greater detail in the non-limiting Examples that follows. (See also Hale et al, Clinical Cancer Res. 7:846 (2001)).

### EXAMPLE 1

ZAG Expression by Malignant Prostatic Epithelium

Experimental Details

Tissue and Serum Samples:

Normal and malignant prostate tissues were used as formalin-fixed, paraffin-embedded (FFPE) sections. To eliminate potential selection bias, all prostatectomy specimens obtained during a 3 month period that had sufficient tumor available for examination were used in this study. This yielded 16 specimens with a combined Gleason sum of 5-6 (moderate grade), 13 specimens with a combined Gleason sum of 7 (borderline high grade), and 3 specimens with a combined Gleason sum of 8-9 (high grade). To obtain additional numbers of high grade tumors for evaluation, all prostatectomy specimens with Gleason sums of 8-9 obtained in the same year were added to the study (total n=19). Blocks that contained tumor as well as residual benign prostatic epithelium were selected for study. Nine additional cases of prostate tissue obtained by transurethral resection of the prostate with no evidence of malignancy were studied as controls. Clinical characteristics of patients from whom samples were obtained are summarized in Table 1. Matched frozen and FFPE samples of normal and prostate cancer tissues obtained anonymously as discarded tissue also were used as controls to verify appropriate antigen retrieval and to optimize immunohistochemical staining.

TABLE 1
Clinical Characteristics of Patients in Case
Series for ZAG Immunohistochemical Staining

| Tumor Grade | Age (years)* | Average Gleason Sum |
|---|---|---|
| No tumor (n = 9) | 71 ± 8 (61-84) | N.A. |
| Moderate (n = 16) | 59 ± 6 (49-70) | 5.6 |
| Borderline High (n = 13) | 65 ± 8 (49-78) | 7.0 |
| High (n = 19) | 67 ± 8 (56-83) | 8.6 |

Serum samples obtained as part of a previous hospital-based prostate cancer case-control investigation aimed at determining anthropometric and hormonal risk factors were used to explore ZAG expression in both malignant prostatic tissue and sera. Methods for this study have been reported elsewhere (Demark-Wahnefried et al, J. Androl. 18:495-500 (1997), Demark-Wahnefried et al, Nutr. Cancer 28:302-307 (1997)). In brief, both cases and controls for this study were weight-stable (<5% change in body weight within one year of study recruitment), had no current or past use of hormonal agents, no history of other cancers (with the exception of non-melanoma skin cancer), and were 50-70 years of age. Cases were ascertained within three months of diagnosis with early stage disease. Eligibility criteria for control patients required normal PSA values and negative digital rectal exams. Sera from this study had been stored at −70° C. and only aliquots from cases that were accrued prior to treatment were accessed for the current study. Additionally, control subjects who subsequently developed cancer (other than non-melanoma skin cancer) within 3 years of original participation were excluded from the current study. Selected serum aliquots were anonymized, coded and analyzed for ZAG in blinded fashion, with two race- and age-matched controls (n=28) selected for every case (n=14) (see Table 2). FFPE tumor samples from each case patient were retrieved from archives and assayed for ZAG via immunohistochemistry. Tumors with detectable ZAG immunoreactivity (score of ≧1) were scored as ZAG-positive.

TABLE 2
Clinical Characteristics of Patients in Case-Control
Series for Serum ZAG Measurements

| | | | | Average |
|---|---|---|---|---|
| | | | Body Mass | Gleason |
| | Race | Age* | Index* | Sum |
| Cases (n = 14) | 13 white | 64 ± 6 (52-70) | 26.6 ± 3.1 | 6.3 |
| | 1 black | | (20.7-33) | |
| Controls (n = 28) | 26 white | 64 ± 5 (51-70) | 27.8 ± 3.2 | N.A. |
| | 2 black | | (21.8-35.1) | |

*Average ± standard deviation (range)
N.A. = not applicable

Immunohistologic Studies:

Immunohistochemical assays were optimized using matched samples of frozen and FFPE tissues to ensure that appropriate immunoreactivity was retained in FFPE tissues. Four micron FFPE sections were stained using standard protocols, including blocking of endogenous peroxidase activity (0.6% H₂O₂ in absolute methanol, 15 min), antigen retrieval with microwave citrate (10 mM sodium citrate, pH 6.0, 2×5 min, 600W) and blocking with 10% horse serum in PBS. The slides were then sequentially incubated at 37° C. with primary anti-ZAG monoclonal antibody 1H4 (Sanchez et al, Proc. Natl. Acad. 94:4626-4630 (1997)), biotinylated secondary antibody, and avidin-biotin-horseradish peroxidase complexes (VectaStainABC, Vector Laboratories, Burlingame, Calif.), with intervening PBS washes. Bound antibody was detected with 3,3′-diaminobenzidine plus H₂O₂. The immunoreactivity of FFPE sections using this protocol was identical to that of frozen tissue, except that nuclear staining was occasionally seen focally in some FFPE tissues. As nuclear staining was never observed in frozen tissues or FFPE tissues treated with enzyme-based antigen retrieval, this occasional focal nuclear staining was clearly an fixation-dependent artifact of the microwave antigen retrieval process, and was ignored. The staining protocol was optimized such that serial sections stained with an equivalent concentration of isotype-matched control antibody showed total lack of color development. Immunohistochemical reactivity of tumors was rated independently by two board-certified pathologists according to the following scale: 0=absence of reactivity in >50% of tumor cells; 1=faint but clearly detectable reactivity in >50% of tumor cells; 2=moderate reactivity in >50% of tumor cells; 3=strong reactivity in >50% of tumor cells. The staining intensity of residual non-apocrine prostate epithelium in each section was assigned a score of 2 to allow normalization. Given that Gleason scores could be assessed at the time the ZAG-immunostained slides were reviewed, true blinding was not possible, however the Gleason sum derived from examination of all slides obtained for each case was not available to the observers at the time the ZAG-immunostained sections were evaluated.

Measurement of Serum ZAG:

Serum ZAG levels were determined by an antigen capture enzyme immunoassay, using anti-ZAG mAb 1B5 (Sanchez et al, Proc. Natl. Acad. Sci. USA 94:4626-4630 (1997)) as capture antibody. Bound ZAG was detected using biotinylated anti-ZAG mAb 1H4, streptavidin-horseradish peroxidase conjugate (Jackson ImmunoResearch Labs, West Grove, PA), and 3,3′,5,5′-tetramethyl-benzidine substrate (Kirkegaard and Perry Laboratories, Gaithersburg, Md.). Standard curves were constructed using recombinant human ZAG, quantitated by A₂₈₀ of HPLC purified ZAG (Burgi et al, J. Biol. Chem. 236:1066-1074 (1961)). Each serum sample was analyzed in quadruplicate for at least 2 independent dilutions and results were averaged. The sensitivity of the assay was 10 pg/ml.

Generation of ZAG-Producing Murine Cell Lines:

A full length human ZAG cDNA including the endogenous secretory signal sequence was cloned from human liver using RT-PCR. The primers used corresponded to bp 3-21 and bp 938-920 (GenBank D90427). The construct sequence was verified by automated DNA sequencing then inserted into the pCDNA3.1(−) Myc-His eukaryotic expression vector (Invitrogen) using restriction enzyme digestion and adapter ligation to ensure in-frame insertion relative to the myc and 6-His 3′ epitope tags. Epitope-tagged human ZAG constructs were transfected into B16 murine melanoma cells and stable transfectants were obtained by G418 selection then cloned by limited dilution.

Selected clones expressed high levels of epitope-tagged human ZAG with the predicted molecular weight of 46 kDa, as verified by antigen capture ELISA and Western blot of culture supernatant.

Animal Studies

2×10⁵ ZAG or vector-transfected B16 tumor cells were implanted subcutaneously in the flank in groups of 5 syngeneic female C57BL/6 mice. Serum was obtained and mice were weighed at 21 days, just prior to tumor-related death. The concentration of tumor-produced human ZAG in the serum was measured by antigen capture ELISA as described above. To address whether tumor-produced ZAG could be detected in the serum when tumors were grown orthotopically within the prostate, CWR22 androgen-dependent human prostate cancer cells suspended in matrigel (Collaborative Research, Bedford, Mass.) at a concentration of 5×10⁶ cells/100 μl were injected orthotopically into the ventral prostate of 6 week old male nude rats (n=9). This orthotopic nude rat model facilitated accurate implantation and growth of a xenogeneic human ZAG-expressing tumor directly within the prostate. Sixty days after surgical implantation animals were euthanized and both serum and tumor were harvested and analyzed for expression of human ZAG.

Statistical Analysis:

To test the association between the ZAG score of immunostained prostate cancer samples and tumor grade, the Mantel-Haenzael correlation statistic with rank scores assigned to both variables was used. The association is described by giving means on ZAG score by grade. To test whether the mean serum ZAG concentration of controls was different from that of cases, a difference score equal to the natural log of the serum ZAG concentration of the prostate cancer case minus the natural log of the serum ZAG concentration of the control was calculated and tested to determine whether the mean of the difference scores was equal to zero for each case/control match. Natural logs were used to successfully approximate normality. Repeated measures analysis via the MIXED procedure using SAS software (SAS Institute Inc., Cary, N.C.) was used to calculate the test statistic, since this procedure allowed the two difference scores for each case to serve as a correlated “cluster.” These matched data also were tested to determine whether the prostate cancer cases had higher serum ZAG concentrations significantly more often than their matched controls. This test was calculated using repeated measures logistic regression via the GENMOD procedure in SAS, to account for the fact each case was matched to two controls.

Results

ZAG is Expressed by Benign Prostate Epithelium, But Not by Seminal Besicles:

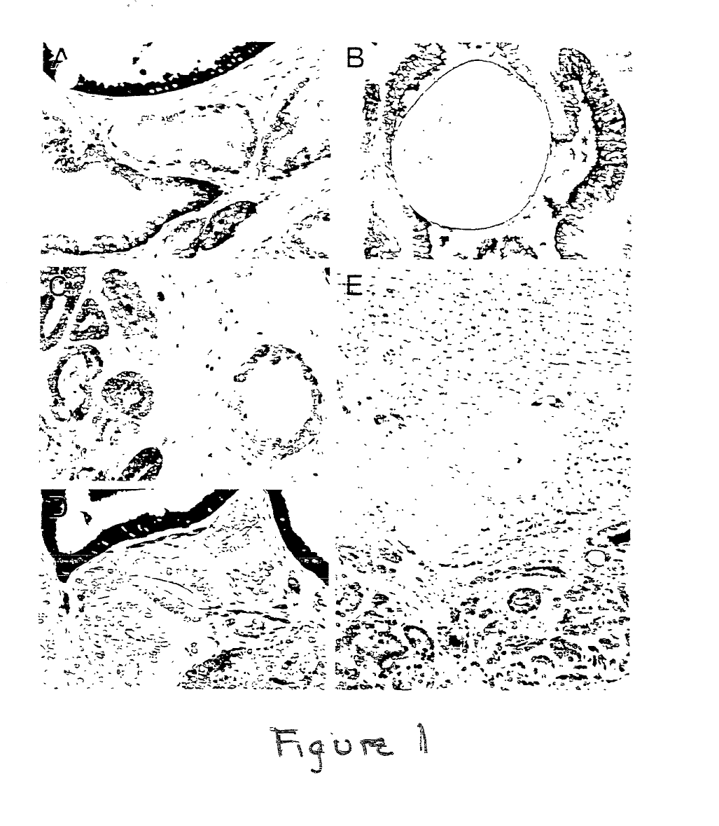

Normal benign epithelium was moderately to strongly reactive with anti-ZAG antibody in 9 of 9 normal human prostates tested. In addition, normal prostate acini present on sections that also contained prostate cancer were similarly moderately to strongly reactive with anti-ZAG antibody (48 of 48 cases; FIG. 1A). The immunohistochemical reactivity of the normal non-apocrine prostate epithelium in each section with anti-ZAG antibody was given a score of 2 to facilitate semi-quantitative comparison of ZAG expression between different prostate cancers (see below). The overall immunoreactivity of normal prostate epithelium correlated with secretory activity, and was highest in dilated glands containing copious luminal apocrine-type secretions (FIG. 1A, top of panel). These highly reactive glands were assigned an immunohistochemical reactivity score of 3. The concretions present in normal acini also were highly reactive with ZAG mAb (FIG. 1B), indicating that ZAG protein is a prominent constituent of these concretions.

Since the concentration of ZAG in seminal fluid has previously been reported to be high (Poortmans et al, J. Lab. Clin. Med. 71:807-811 (1968)), a determination was made of the cellular source of seminal fluid ZAG by immunohistochemical comparison of prostate and seminal vesicle tissues. No evidence of ZAG immunoreactivity was found in any of the 11 seminal vesicles studied. The prostatic duct also was non-reactive with anti-ZAG antibody. The high levels of ZAG immunoreactivity seen in normal prostate, taken together with the total absence of ZAG immunoreactivity in seminal vesicle and associated ducts, demonstrates that the ZAG previously described to be present in seminal fluid (Poortmans et al, J. Lab. Clin. Med. 71:807-811 (1968)) must be produced by the epithelium of the prostate itself.

Prostate Carcinomas React with ZAG mAb:

35 of the 48 (73%) of the prostate cancers studied were reactive with anti-ZAG antibody (Table 3). The pattern of ZAG immunoreactivity in positive tumors varied from global cytoplasmic staining (FIG. 1C) to strong staining only on the luminal surface (FIG. 1D). In some tumors, there were local variations in the intensity of ZAG immunoreactivity, but usually with clear boundaries that suggested discrete tumor subpopulations. For example, one well-defined tumor nodule might be strongly positive with an adjacent tumor nodule only weakly positive (FIG. 1E) or even negative. As shown in Table 3, the intensity of immunostaining with anti-ZAG antibody also varied among tumors with similar Gleason scores. However, high grade tumors were significantly more likely to be ZAG-negative or to have decreased ZAG immunostaining relative to moderate grade tumors. The Mantel-Haenzel test of the association between ZAG and tumor grade gave a p-value of 0.01 for a mean ZAG scores of 1.1 for high grade (Gleason sum 8-9) vs. 1.7 for borderline high (Gleason sum 7) vs. 1.9 for moderate grade (Gleason sum 5-6) tumors.

TABLE 3
Reactivity of Prostate Cancers
with anti-ZAG Antibody

| | ZAG Score* | | | |
|---|---|---|---|---|
| Tumor Grade | 0 | 1 | 2 | 3 |
| Moderate (n = 16) | 6% (n = 1) | 25% (n = 4) | 38% (n = 6) | 31% (n = 5) |
| Gleason score 5-6 | | | | |
| Borderline High (n = 13) | 23% (n = 3) | 16% (n = 2) | 31% (n = 4) | 31% (n = 4) |
| Gleason score 7 | | | | |
| High (n = 19) | 47% (n = 9) | 16% (n = 3) | 26% (n = 5) | 11% (n = 2) |
| Gleason score 8-9 | | | | |
| Totals | 27% n = 13 | 19% n = 9 | 31% n = 15 | 23% n = 11 |

*ZAG score was derived as described above. 0 = absence of reactivity in >50% of tumor cells; 1 = faint but clearly detectable reactivity in >50% of tumor cells; 2 = moderate reactivity in >50% of tumor cells; 3 = strong reactivity in >50% of tumor cells. The staining intensity of residual non-apocrine prostate epithelium in each section was normalized to a score of 2.

Prostate tissues in which tumor cells demonstrated strong ZAG immunoreactivity also showed increased immunostaining of tumor-associated and benign stroma (FIG. 1C). These regions did not show increased background staining with isotype-matched control antibody. Therefore, increased immunostaining most likely represents detection of tumor-produced ZAG that has “spilled out” into the adjacent stroma. Unlike normal prostatic concretions (FIG. 1B), malignant crystalloids were non-reactive with ZAG mAb.

Serum ZAG Levels Increase in Patients with ZAG-Positive Prostate Cancers:

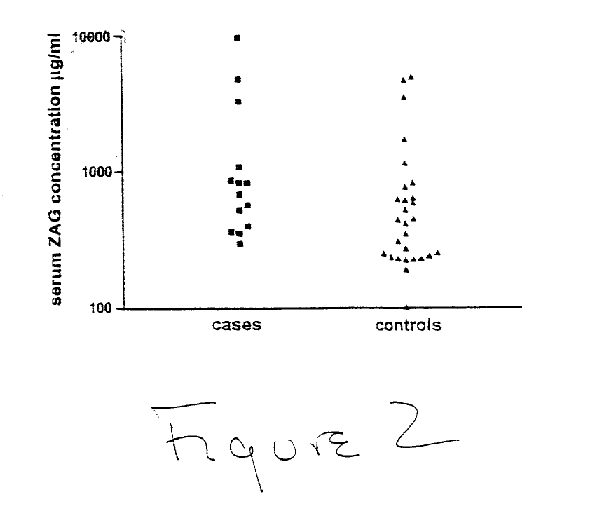

To determine whether ZAG production in tumors was associated with an increased serum concentration of ZAG, serum ZAG concentrations were analyzed in a cohort of patients with documented prostate cancer (n=14) and age- and race-matched controls (n=28) using an antigen capture immunoassay. Eleven of 14 cancer patients had ZAG-positive tumors (ZAG score of ≧1) by immunohistochemistry. Two of the 3 tumors with ZAG scores of 0 had small foci with faint ZAG staining but did not meet the 50% area requirement for ZAG positivity. Thus, 13 of 14 patients with prostate cancer had at least some ZAG production by cancer cells. Serum ZAG concentrations obtained for both patients and controls are shown in FIG. 2. The test of a mean difference in serum ZAG concentration between cases and controls gave a p-value of 0.10. The test of whether prostate cancer cases had higher serum ZAG concentrations significantly more often than the controls to which they were matched gave a p-value of 0.02; of the 28 matched pairs, the cases had the larger value 20 times. Clinical follow-up revealed that, although all controls had a negative digital rectal exam and normal PSA values at enrollment, 4 of the 28 control patients had biopsies demonstrating the presence of prostate carcinoma within 3-7 years after serum donation. The serum ZAG levels of these 4 patients averaged 579 μg/ml (range 243-826) in the present study. While larger studies with long term follow-up are needed, it appears that elevated serum ZAG levels occur early in prostate cancer progression, prior to its detectability by digital rectal exam or elevated PSA.

Tumor-Produced ZAG Contributes to Serum ZAG Levels in Murine Models:

To definitively test the hypothesis that tumor-produced ZAG contributes to an elevated concentration of circulating ZAG, it was necessary to generate a model system in which tumor-produced ZAG could be differentiated from ZAG produced by normal secretory epithelia. Murine tumor cell lines expressing epitope-tagged recombinant human ZAG were therefore produced that could be specifically identified and distinguished from endogenous murine ZAG produced by normal secretory epithelium by using antibodies that recognize either human ZAG or the epitope tag, but do not cross-react with murine ZAG. Human ZAG could be detected in the serum of mice bearing hZAG-transfected B16 tumors (156+70 ng/ml; n =5), but not in the serum of mice bearing vector-transfected B16 tumors (n=5), when the tumor was implanted in a subcutaneous location. This level of increased ZAG production was sufficient to cause mean weight loss of 15% in the group bearing hZAG-transfected tumors (ending weights: B16-vector 20.6+1.1 g; B16-ZAG 17.5+0.8 g; p =0.001).

To show that human prostate carcinomas growing orthotopically within the prostate could similarly contribute to elevated serum ZAG levels, the ZAG-producing CWR22 human prostate carcinoma was implanted directly into the prostate of nude rats. As in the murine model described above, tumor-produced hZAG is readily distinguished from endogenous rat ZAG in this model using antibodies specific for hZAG that do not cross-react with rat ZAG. Rats with intraprostatic CWR22 tumors had 59±24 ng/ml hZAG present in their serum (mean ±SD, n=7), while two rats in which tumors were implanted but failed to grow had undetectable serum levels of hZAG.

### EXAMPLE 2

Role of ZAG in Tumor-Associated Thymic Atrophy

1. Marked Atrophy of the Thymus Occurs in Tumor-Bearing Animals.

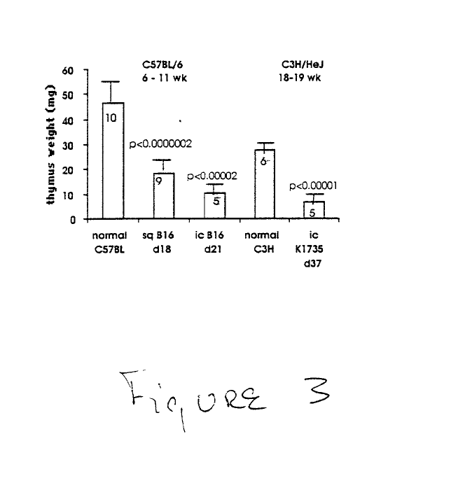

The phenomenon of tumor-associated thymic atrophy was independently rediscovered when thymus weights from mice bearing tumors were compared with age-matched non-tumor-bearing control mice. B16 melanoma tumors were implanted into syngeneic C57BL/6 mice in both subcutaneous (SQ) and intracranial (IC) locations. Mice were sacrificed on day 18 (SQ) or day 21 (IC), and thymus weights were determined. Thymus weights from C3H/HeJ mice bearing the syngeneic K1735 melanoma tumor intracranially were similarly measured on day 37 after implantation. It was found that thymus weights were significantly decreased (p<0.00002) in mice bearing B16 tumors, in either SQ or IC locations (FIG. 3). Thymus weights in mice bearing K1735 tumors were also significantly decreased relative to non-tumor-bearing controls (p<0.00001). After weighing the entire thymus, a portion was removed, weighed again, and thymocytes were obtained by pressing the tissue gently through a mesh screen. Cell counts were obtained and absolute thymocyte counts for the entire thymus were calculated. Cells were then stained with directly labeled CD4 and CD8 antibodies and analyzed by flow cytometry. The total number of cells per thymus correlated with thymus weights, with significant decreases in cellularity observed in thymuses with decreased overall weight. Decreases were seen in all thymocyte subsets, with the largest % decrease seen in immature CD4+CD8+(double positive, DP) thymocytes.

2. Tumor Production of ZAG is Associated with Thymic Atrophy in Mice.

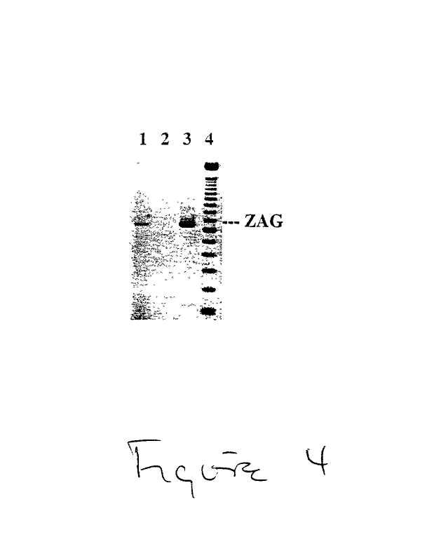

Marked thymic atrophy was observed in the studies of tumor-bearing mice under conditions where the tumor remained localized and distant from the thymus, suggesting that thymic atrophy resulted directly or indirectly from factor(s) produced by tumor and delivered to the thymus or adjacent tissue via the circulation. Thymic atrophy has previously been described to occur during starvation (Dourev, Curr. Topics Pathol. 75:127 (1986)) and in other conditions where stored body fat is utilized, including hibernation (George et al, Immunol. Today 17:267 (1996)). It was questioned whether a lipolytic state induced by tumor-produced ZAG could potentially play a role in tumor-associated thymic atrophy. Therefore, an analysis was made of the B16 and K1735 tumors that had already shown induced tumor-associated thymic atrophy (FIG. 3) for ZAG mRNA production using RT-PCR. Both B16 and K1735 tumors induce marked thymic atrophy and produce abundant ZAG mRNA (FIG. 4).

A screen of other murine tumor cell lines by RT-PCR identified the 4T1 murine breast carcinoma that does not make ZAG mRNA. Five Balb/C female mice (12 weeks old) were implanted with 1×10⁶ syngeneic 4T1 tumor cells subcutaneously in the flank. Mice were sacrificed at day 20, just prior to natural death from tumor. Mice bearing 4T1 (ZAG-negative) tumors showed no change in either body weight or thymus weight as compared to non-tumor-bearing mice (body weights +SEM: 19.5±0.6 g (4T1) vs. 19.6±10 g (control), p=0.93; thymus weights ±SEM: 47.6±8.4 mg (4T1) vs. 42.1±5.0 mg (control), p=0.60). These studies further indicate that ZAG plays a role in tumor-associated thymic atrophy.

3. Generation of Recombinant Human and Murine Epitope-Tagged ZAG Constructs and Purification of Recombinant Human ZAG.

To further investigate the hypothesis that ZAG is involved in tumor-associated thymic atrophy, full length ZAG cDNAs were cloned from both human and murine liver using RT-PCR. Primers used for cloning human (h) ZAG corresponded to bp 3-21 and bp 938-920 (GenBank D90427). Primers used for cloning murine (m) ZAG corresponded to bp 1-18 and bp 1036-1015 (GenBank D21059). The sequence of each construct was verified by automated DNA sequencing. Each construct was then inserted into the pCDNA3.1(−) Myc-His vector for expression in eukaryotic cells. The endogenous secretory signal sequence was used in these constructs. Restriction enzyme digestion and adaptor ligation were used to ensure in-frame insertion relative to the 3′ epitope tags.

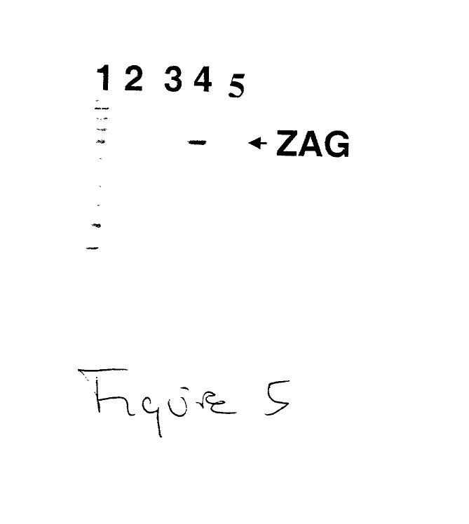

The use of hZAG was focussed on since hZAG has been shown to function similarly to mZAG in mice (Hirai et al, Cancer Res. 58:2359 (1998)), since 9 mAbs were available that recognize hZAG (received from Dr. Luis Sanchez; Sanchez et al, Proc. Natl. Acad. Sci. 94:4626 (1997)), and most importantly since the use of hZAG in mice makes it easy to distinguish tumor-produced ZAG from endogenous murine ZAG normally produced by secretory epithelial tissues that will not react with hZAG mAbs. Epitope-tagged recombinant hZAG (rhZAG) constructs were transfected into the 293 human kidney epithelial cell line and stable transfectants were obtained using G418 selection. The rhZAG construct drives the secretion of an epitope-tagged protein of the expected MW (FIG. 5). Milligram quantities of rhZAG were purified from spent supernatant of these cells using a combination of affinity chromatography and HPLC for use as standards in ELISA assays and for future functional studies.

4. Creation of Murine Tumor Cell Lines Differing Only in ZAG Production.

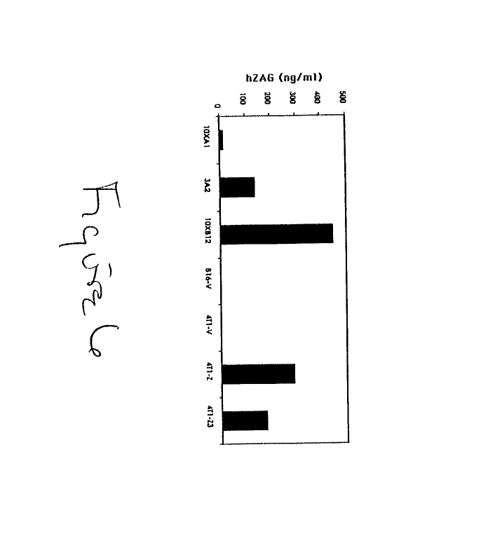

To directly address the role of ZAG in tumor-associated thymic atrophy, several murine cell lines stably transfected with recombinant human (rh) ZAG constructs were derived and characterized. These include the B16 melanoma cell line transfected with rhZAG, vector alone, and mouse antisense-ZAG. These cell lines represent very high, moderate, and low expression of total (mouse+human) ZAG, respectively in a tumor type that have already shown to induce thymic atrophy. In addition to these lines, B16-rhZAG clones were selected that secrete low, moderate, and high amounts of rhZAG for potential use in dose-response studies to determine the role of tumor-produced ZAG in thymic atrophy (FIG. 6). 4T1 (murine ZAG-negative) cells stably transfected with vector alone and with the rhZAG construct (FIG. 6) were also derived. Northern blots of RNA and Western blots of culture supernatant were analyzed from the transfected populations, confirming the production of both ZAG mRNA and protein by these transfected cell lines. rhZAG secretion was documented by antigen capture enzyme immunoassay. ZAG-specific mAb 1B5 bound to microtiter plates was used to capture rhZAG present in culture supernatants. Captured rhZAG was then detected using biotinylated anti-ZAG mAb 1H4 (Sanchez et al. Proc. Acad. Sci. 94:4626 (1997)).

5. rhZAG is Biologically Active and Causes Thymic Atrophy in Tumor-Bearing Mice.

To directly test the hypothesis that ZAG protein secreted by tumors causes tumor-associated thymic atrophy, ZAG-negative 4T1 parent and rhZAG-transfected 4T1 breast carcinoma tumor cells, were implanted into groups of 5 syngeneic Balb/C female mice. Mice were sacrificed when tumors reached 1.0 cm³. Data are summarized in Table 4. Thymus weights were markedly decreased in mice with 4T1-rhZAG tumors, with corresponding decreases in absolute numbers of thymocytes per thymus. ZAG transfection did not affect tumor growth.

Mice bearing 4T1-rhZAG tumors failed to gain weight during the study, amounting to a significant weight oss for mice bearing tumors expressing rhZAG after body weights were corrected for tumor weight. This confirms the biologic activity of the rhZAG in mice in vivo.

TABLE 4
ZAG Transfection is Sufficient to Induce Thymic Atrophy in Mice

| | 4T1 parent (ZAG) | 4T1-rhZAG |
|---|---|---|
| Thymus weight | 31.1 ± 2.0 mg | 24.0 ± 6.1 mg |
| Number of thymocytes, ×10⁶ | 92 ± 21 | 54 ± 23 |
| Tumor volume | 1257 ± 583 mm³ | 1043 ± 112 mm³ |
| Change in body weight | +1.3 g | +0.1 g |

Values given are mean ±SD for 5 animals studied.

All documents cited above are hereby incorporated in their entirety by reference.

What is claimed is:

1. A method of diagnosing cancer in a test mammal comprising assaying for the level of zinc α-2-glycoprotein (ZAG) present in a biological sample from said test mammal and comparing that level to a biological sample from a control, non-tumor bearing mammal, wherein an elevated level of ZAG in the biological sample from said test mammal relative to said control is indicative of the presence of a tumor.

2. The method according to claim 1 wherein said biological sample is a liquid sample.

3. The method according to claim 2 wherein said biological sample is a plasma, urine, cerebrospinal fluid, seminal fluid, sweat or nipple aspirate sample.

4. The method according to claim 1 wherein the level of ZAG is assayed using an immunoassay, chromatography, electrophoresis, or solid phase affinity or densitometry of a Western blot.

5. The method according to claim 4 wherein the level of ZAG is assayed using an antigen capture or competitive immunoassay.

6. The method according to claim 1 wherein said method is a method of diagnosing prostate cancer.

7. The method according to claim 6 wherein said biological sample is a serum sample.

8. A method of diagnosing an inflammatory disease or disorder in a test mammal comprising assaying for the level of zinc α-2-glycoprotein (ZAG) present in a biological sample from said test mammal and comparing that level to a biological sample from a control mammal, wherein an elevated level of ZAG in the biological sample from said test mammal relative to said control is indicative of the presence of an inflammatory disease or disorder.

9. The method according to claim 8 wherein said biological sample is a liquid sample.

10. The method according to claim 9 wherein said biological sample is a serum sample.

11. The method according to claim 8 wherein the level of ZAG is assayed using an immunoassay, chromatography, electrophoresis, or solid phase affinity or densitometry of a Western blot.

12. The method according to claim 11 wherein the level of ZAG is assayed using an antigen capture or competitive immunoassay.

13. The method according to claim 8 wherein said inflammation is of the breast, prostate, liver, or salivary, bronchial, gastrointestinal or sweat gland of said mammal.

14. A method of inhibiting thymic atrophy in a mammal comprising administering to said mammal an amount of an agent that reduces the bioavailability of ZAG or that inhibits the binding of ZAG to its receptor sufficient to effect said inhibition of atrophy.

15. The method according to claim 14 wherein said mammal is an adult bearing a tumor.

16. The method according to claim 15 wherein said mammal is undergoing cancer chemotherapy.

17. The method according to claim 14 wherein said mammal has an infection.

18. The method according to claim 17 wherein said infection is an HIV infection.

19. The method according to claim 14 wherein said agent is an anti-ZAG antibody or an antiandrogen.
